(19) United States
(12) Patent Application Publication
(10) Pub. No.: US 2003/0036082 A1
(43) Pub. Date: Feb. 20, 2003

(54) Screening methods

(76) Inventors: Cecile Martijn, Sundbyberg (SE); Eva Rupp-Thuresson, Balsta (SE); Harriet Wallberg-Henriksson, Lidingo (SE); Juleen R. Zierath, Lidingo (SE)

Correspondence Address: FISH & RICHARDSON PC, 225 FRANKLIN ST, BOSTON, MA, 02110, US

(21) Appl. No.: 10/188,648

(22) Filed: Jul. 2, 2002

Related U.S. Application Data
(60) Provisional application No. 60/304,683, filed on Jul. 11, 2001.

(30) Foreign Application Priority Data
Jul. 3, 2001 (SE) 0102385-2

(51) Int. Cl. C12Q 1/68; G01N 33/567

(52) U.S. Cl. 435/6; 435/7.21

(57) ABSTRACT

The present invention relates to the use of diacylglycerol kinase delta (DGKδ) in methods for the identification of pharmaceutically useful agents, in particular agents useful for increasing insulin-stimulated glucose uptake in a mammalian cell and for the treatment or prophylaxis of diabetes.

### CROSS REFERENCE TO RELATED APPLICATIONS

This application claims priority from Swedish Patent Application No. 0102385-2, filed Jul. 3, 2001, and U.S. Provisional Patent Application Serial No. 60/304,683, filed Jul. 11, 2001. These applications are incorporated herein by reference in their entirety.

### TECHNICAL FIELD

The present invention relates to the use of diacylglycerol kinase delta (DGKδ) in methods for the identification of pharmaceutically useful agents, in particular agents useful for the treatment or prophylaxis of diabetes.

### BACKGROUND ART

Diacylglycerol (DAG) functions in intracellular signaling pathways as an allosteric activator of protein kinase C (PKC). DAG also occupies a central position in the synthesis of major phospholipids and triacylglycerols. Thus, to maintain cellular homeostasis, intracellular DAG levels must be tightly regulated. DAG kinases (DGKs or DAGKs; EC 2.7.1.107) phosphorylate DAG to phosphatidic acid, thus removing DAG. Consequently, diacylglycerol kinase is one of the key enzymes involved in the regulation of signal transduction (for a review, see e.g. Topham & Prescott (1999) J. Biol. Chem. 274: 11447-11450). It attenuates, for example, protein kinase C activity and cell cycle progression of T-lymphocytes, through controlling the intracellular levels of the second messengers, diacylglycerol and phosphatidic acid. To date, eight DGK isozymes containing characteristic zinc finger structures in common have been identified.

Sakane et al. (J. Biol. Chem. 271: 8394-8401, 1996) used PCR with degenerated primers to isolate a novel DGK, which they termed DGK-delta, from human testis mRNA. They then cloned full-length cDNAs from human hepatoma and testis cDNA libraries. Sequence analysis revealed that this gene encodes a 1,169-amino acid protein containing, in addition to sequences homologous to other DGKs, a pleckstrin homology domain and a C-terminal tail similar to those of the EPH family of receptor tyrosine kinases. Sakane et al. suggested that, because of its different structural features, this novel DGK belongs to a subfamily of DGKs distinct from the alpha, beta, and gamma DGK isoforms. Northern blot analysis showed that this gene encodes a 6.3-kb transcript most abundant in skeletal muscle but undetectable in brain, thymus, and retina. Sakane et al. expressed this novel DGK gene in COS-7 cells and observed an increase in DGK activity upon overexpression. The measured activity was independent of the presence of phosphatidylserine.

The DNA and amino acid sequences of human DGKδ are publicly available (see, e.g., GenBank T Accession Nos. D63479 and D73409; SwissProt Q16760). The entire content of these DNA and amino acid sequences are incorporated herein by reference.

It has been shown (Nobe et al. (1998) Cell. Signal. 10: 465-471) that basal resting level of DGK activity changed in aorta and kidney isolated from diabetic rats. These changes in DGK activity were said to resemble the functional changes associated with complications of diabetes, suggesting that changes in PI turnover followed by DGK activity are a key element in the complications. DGK activity changes in the tissues described here (aorta and kidney) did not suggest a role for specifically DGKδ, since the delta isoform is not expressed to higher levels in these tissues.

### BRIEF DESCRIPTION OF THE DRAWINGS

FIG. 1 is a graph depicting expression of DGKδ in muscle biopsies quantified by Real-Time PCR. Subjects 1 and 2 are control subjects, while subjects 3 and 4 are diabetic patients. The expression levels are shown in arbitrary units.

FIG. 2 depicts DGKδ protein expression in skeletal muscle. The upper part of the figure shows DGKδ bands, visualized by immunoblotting, from NIDDM patients (D) and control subjects (C). Data are reported as arbitrary optical density units for 4 subjects in each group. The star (*) indicates significance according to Student's unpaired t-test.

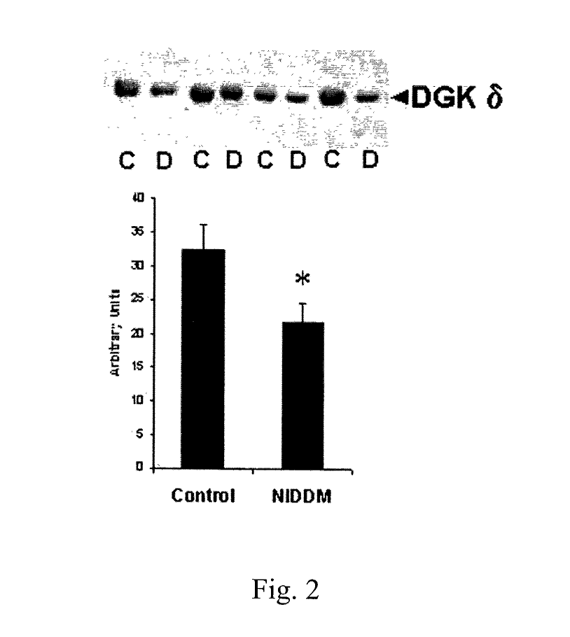

FIG. 3 is a graph depicting DGKδ expression in gastrocnemius muscle from Wistar (W) and GK rats after treatment with phlorizine (phl). Data are reported as arbitrary optical density units. The upper part of the figure shows corresponding DGKδ bands visualized by immunoblotting.

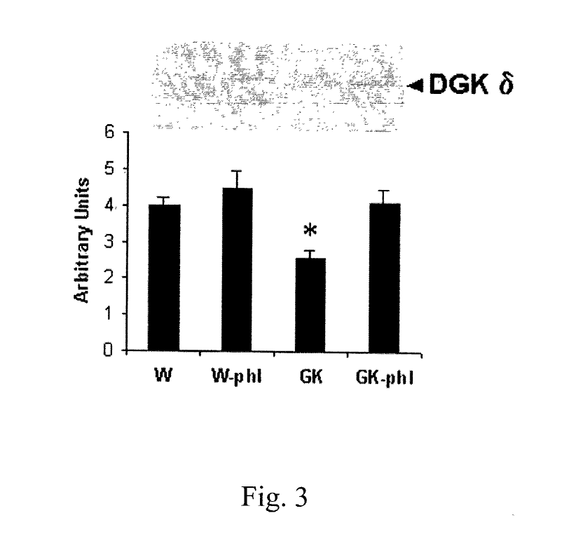

FIG. 4 depicts total DGK activity in skeletal muscle from Wistar (W) and GK rats after treatment with phlorizine (phl). Data are reported as arbitrary optical density units. The upper part of the figure shows corresponding phosphatidic acid bands.

FIG. 5 is a graph depicting glucose transport in the absence (Bas) or presence (Ins) of insulin; and with or without DGK inhibitor II™ (Inh), in isolated trochlearis muscle from Wistar rat. Glucose transport is expressed as μmol per milliliter of intracellular water per hour. Data are reported as mean±SEM for 11-13 animals in each group.

### DISCLOSURE OF THE INVENTION

It has surprisingly been found that DGKδ is useful in methods for the identification of pharmaceutically useful agents, in particular agents useful for the treatment or prophylaxis of diabetes. Expression of DGKδ has been found to be decreased in diabetic patients as well as in a diabetic animal model. Therefore, increasing the activity of DGKδ by pharmaceutically useful agents allows for the treatment or prophylaxis of diabetes.

Consequently, in a first aspect this invention provides a method for identifying an agent useful for the treatment or prophylaxis of diabetes, the method comprising: contacting a candidate agent with a mammalian DGKδ polypeptide; and determining whether the candidate agent activates one or more biological activities of the mammalian DGKδ polypeptide, such activation being indicative for a compound useful for the treatment or prophylaxis of diabetes.

Biological activities of mammalian DGKδ include, for example, insulin-stimulated glucose uptake, reversion of hyperglycemia, and increased insulin sensitivity. Consequently, the invention also provides a method for identifying an agent useful for increasing insulin-stimulated glucose uptake in a mammalian cell, the method comprising: contacting a candidate agent with a mammalian DGKδ polypeptide; and determining whether said candidate agent increases insulin-stimulated glucose uptake in the said mammalian cell.

The method can include a step of determining whether the candidate agent binds to the DGKδ polypeptide. Such a binding detection step can be carried out in vivo or in vitro (e.g., using a cell free assay).

Thus for screening purposes, an enzymatic activity of DGKδ, e.g., recombinantly produced DGKδ, can be determined by known methods (Sakane et al. (1991) J. Biol. Chem. 266, 7096-7100). In some examples, agents increasing enzyme activity will be considered as hits and pursued further. This described increase in enzymatic activity (e.g., kinase activity) can lead to an increase in biological activity, e.g., increase in glucose uptake and improvement of hyperglycemia.

In another aspect, the invention provides a method for identifying an agent useful for the treatment or prophylaxis of diabetes, the method comprising: contacting a candidate agent with a nucleic acid molecule encoding mammalian DGKδ; and determining whether said candidate agent activates the expression of the nucleic acid molecule encoding mammalian DGKδ, such activation being indicative for a compound useful for the treatment or prophylaxis of diabetes.

The method can also include a step of determining the effect of the candidate agent on insulin-stimulated glucose uptake in a cell.

For screening purposes, appropriate host cells can be transformed with a vector having a reporter gene under the control of DGKδ. The expression of the reporter gene can be measured in the presence or absence of an agent with known activity (i.e. a standard agent) or putative activity (i.e. a “test agent” or “candidate agent”). A change in the level of expression of the reporter gene in the presence of the test agent is compared with that effected by the standard agent. In this way, active agents are identified and their relative potency in this assay determined.

A transfection assay can be a particularly useful screening assay for identifying an effective agent. In a transfection assay, a nucleic acid containing a gene such as a reporter gene that is operably linked to a DGKδ promoter, or an active fragment thereof, is transfected into the desired cell type. A test level of reporter gene expression is assayed in the presence of a candidate agent and compared to a control level of expression. An effective agent is identified as an agent that results in a test level of expression that is different than a control level of reporter gene expression, which is the level of expression determined in the absence of the agent. Methods for transfecting cells and a variety of convenient reporter genes are well known in the art (see, for example, Goeddel (ed.), Methods Enzymol., Vol. 185, San Diego: Academic Press, Inc. (1990); see also Sambrook, supra).

As used herein, the term “reporter gene” means a gene encoding a gene product that can be identified using simple, inexpensive methods or reagents and that can be operably linked to DGKδ or an active fragment thereof. Reporter genes such as, for example, a luciferase, β-galactosidase, alkaline phosphatase, or green fluorescent protein reporter gene, can be used to determine transcriptional activity in screening assays according to the invention (see, for example, Goeddel (ed.), Methods Enzymol., Vol. 185, San Diego: Academic Press, Inc. (1990); see also Sambrook, supra).

The phrase “nucleic acid molecule encoding mammalian DGKδ” should be understood as including non-translated regions of such nucleic acid molecules. For instance, 5′-prime non-translated regions could be useful in methods for identifying RNA-binding molecules, as described in Swedish patent application No. 0101218-6, filed on Apr. 5, 2001. Such methods comprise the steps

(a) predicting the structure of an RNA-fragment;

(b) choosing a suitable predicted RNA-fragment of step (a), which RNA-fragment comprises at least one individual stem;

(c) synthesizing the DNA-fragment corresponding to the RNA-fragment of step (b);

(d) inserting the DNA-fragment of step (c) in the upstream proximity of a reporter assay gene, which reporter assay gene produces a signal upon translation, thereby forming a reporter construct;

(e) performing a reporter gene assay, which assay monitors the interaction between a molecule to be tested for RNA-binding and the RNA-fragment of the reporter construct.

In a further aspect, the invention provides a method for identifying a modulator of insulin-stimulated glucose uptake, the method comprising: providing a cell expressing a recombinant DGKδ polypeptide; exposing the cell to a candidate agent; and measuring insulin-stimulated glucose uptake in the cell in the presence of the candidate agent, wherein altered insulin-stimulated glucose uptake in the cell in the presence of the candidate agent compared to the absence of the candidate agent indicates that the candidate agent is a modulator of insulin-stimulated glucose uptake.

In a further aspect, the invention provides a method for modulating insulin-stimulated glucose uptake in a cell, the method comprising contacting a cell with an amount of a compound effective to modulate expression or activity of a DGKδ polypeptide and thereby modulate insulin-stimulated glucose uptake in the cell. In one example, the compound increases expression of the DGKδ polypeptide. In another example, the compound increases kinase activity of the DGKδ polypeptide.

In a further aspect, the invention provides a method for the treatment or prophylaxis of diabetes, comprising administering to a subject an effective amount of an agent identified by a method described herein.

In one embodiment, the invention features a method for treating or preventing diabetes, the method comprising: selecting an individual that has or is at risk of having diabetes; and administering to the individual a compound that increases expression or activity of a DGKδ polypeptide in an amount effective to treat or prevent diabetes. For example, the method can include a step of diagnosing the individual as having diabetes.

In yet a further aspect, the invention provides a method for diagnosis of a predisposition to diabetes in a mammalian subject, comprising detecting the level of DGKδ expression in a cell or tissue derived from the said mammalian subject, a decreased level of DGKδ expression, as compared to a healthy subject, being an indication of said predisposition to diabetes.

In one embodiment, the invention features a method of diagnosing the presence or risk of diabetes in a mammalian subject, the method comprising: detecting the level of DGKδ expression in a cell or tissue derived a mammalian subject; and comparing the level of DGKδ expression to a healthy subject, wherein a decreased level of DGKδ expression, as compared to a healthy subject, indicates the presence or risk of diabetes in the mammalian subject. In one example, the method includes detecting the level of a nucleic acid encoding a DGKδ polypeptide. In another example, the method includes detecting the level of a DGKδ polypeptide.

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Below, the invention is described in the appended examples, which are intended to illustrate the invention, without limiting the scope of protection.

### EXAMPLES

### Example 1

### DGKδ RNA Expression Levels are Decreased in Diabetic Patients

(a) Array Experiments

Healthy volunteers and diabetic patients were matched for age, BMI and physical fitness as described in Krook et al, (2000) Diabetes 49: 284-292. RNAs were extracted from muscle biopsies from the diabetic patients and the control subjects. RNAs were reverse transcribed using a T7-tagged oligo-dT primer and double-stranded cDNAs were generated. The cDNAs were amplified and labeled using In Vitro Transcription (IVT) with T7 RNA polymerase and biotinylated nucleotides. The populations of cRNAs obtained after IVT were purified and fragmented by heat to produce a distribution of RNA fragment sizes from approximately 35 to 200 bases. Affymetrix GeneChip® expression arrays (Hu6800, Human expression analysis probe array) were hybridized (using the recommended buffer) overnight at 45° C. with the samples. The arrays were washed and stained with R-phycoerythrin streptavidin with the help of Affymetrix Fluidics Station. The cartridges were scanned using Hewlett-Packard confocal scanner and the images were analyzed with the GeneChip® 3.1 software (Affymetrix) to identify genes differentially expressed between diabetic patients and control subjects. The level of expression of DGKδ (GenBank Accession No. D63479) was decreased 2.5-fold in the diabetic patients as compared to the control subjects.

(b) Quantitative PCR Experiments

Quantitative PCR was performed with real-time TaqMan® and the ABI Prism® 7700 Sequence Detection System (PE Applied Biosystems). DGKδ was quantified based on a standard curve from total RNA and normalized to the endogenous 18S ribosomal RNA gene. Total RNA was isolated from muscle biopsies by using the Rneasy kit (Qiagen). Five hundred nanograms of RNA were reverse transcribed into cDNA using the TaqMan® reverse transcription kit (PE Applied Biosystems) following the manufacturer's instructions. After synthesis, quantitative PCR was performed with 15 ng cDNA in a 25 μl reaction containing 1×TaqMan® PCR buffer, 200 μM dATP, dCTP, dGTP, and dUTP, 5 mM MgCl₂, 0.625 U of AmpliTaq Gold DNA polymerase, 300 nM of each primer set, and 200 nM of a gene-specific detection probe. The detection probe contains two fluorescent dyes, a reporter dye, FAM, and a quenching dye, TAMRA. The cycling conditions included a hot start for 10 min at 95° C. followed by 40 cycles of 95° C. for 30 seconds and 60° C. for 1 min. All reactions were performed in ABI Prism 7700 and data was analyzed using a Sequence Detection System software 1.6.3 (PE Applied Biosystems).

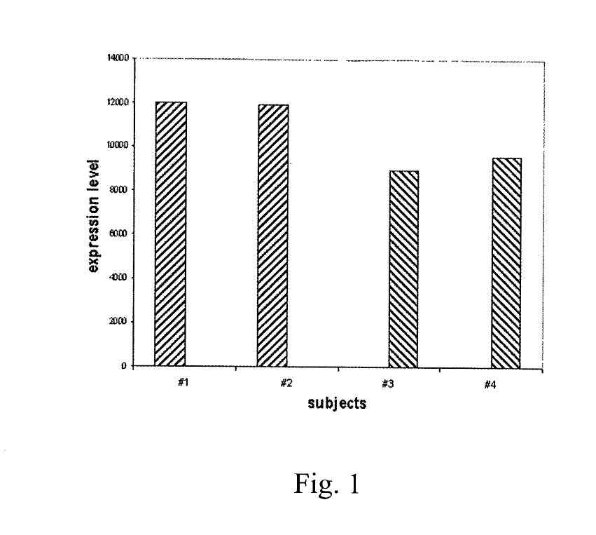

The results (FIG. 1) show that the level of expression of DGKδ mRNA in skeletal muscle from diabetic patients is reduced by 36% as measured by real-time PCR.

### Example 2

### DGKδ Protein Expression Levels are Decreased in Diabetic Patients

DGKδ protein expression levels in vastus lateralis from healthy volunteers and diabetic patients was determined by immunoblotting. Muscle lysates were prepared as described in Krook et al. (2000) Diabetes 49: 284-292. 75 μg protein were separated by SDS-PAGE, transferred to PVDF membranes, and blocked overnight (5% milk in Tris-buffered saline with 0.1% Tween-20). The membranes were then incubated with DGKδ-specific antibodies (Sakane et al. (1996) J. Biol. Chem. 271: 8394-8401) at 4° C. overnight and washed in Tris-buffered saline with 0.1% Tween-20. Bound antibodies were detected with horse radish peroxidase-linked goat anti-rabbit IgG (1:25000; BioRad Laboratories, CA), incubated at room temperature for 1 h. DGK isoforms were visualized by enhanced chemiluminescence (Amersham, Arlington Heights, Ill.) and quantified by densitometry.

### Example 3

### Phlorizin Increases DGKδ Activity in a Diabetic Animal Model

In order to investigate the influence of DGKδ on insulin-stimulated glucose uptake in more detail, DGKδ expression studies and measurements of DGK activity were performed in GK rats, a lean animal model for type 2 diabetes (Goto et al., pp. 301-303 in: Frontiers in Diabetes Research (Shafrir E., Renold, A E, Eds.) London, John Libbey, 1988). Male GK rats (200-250 g) were obtained from the Karolinska Institute, Sweden. Weight-matched male Wistar rats served as controls (B&K Universal, Sollentuna, Sweden). All rats were maintained under a 12-hour light/dark cycle, and had free access to water and standard rodent diet. Four groups of animals were studied: vehicle-treated Wistar rats (n=5), phlorizin-treated Wistar rats (n=6), vehicle-treated GK-rats (n=6) and phlorizin-treated GK-rats (n=6). Phlorizin (0.8 g/kg body wt per day; as 40% solution in propylene glycol) or vehicle (equal amounts of propylene glycol per kilogram) was administered as subcutaneous injection in equal doses at 12-h intervals for 4 weeks. Levels of plasma insulin and glucose tolerance in treated and untreated Wistar and GK rats have previously been reported (Krook et al. (1997) Diabetes 46: 2110-2114).

(a) DGKδ Expression

Diabetic GK rats show an increased level of blood glucose. Treatment with phlorizin reverts this phenomenon and brings glucose levels back to normal values reported (Krook et al. (1997) Diabetes 46: 2110-2114). In order to investigate a potential influence of DGK on glucose transport, DGK expression levels were measured in GK rats, treated with phlorizin or untreated, as well as in normal, healthy Wistar rats.

Portions of the gastrocnemius skeletal muscle (approximately 30 mg) were crushed in liquid N₂ and then lysed in ice-cold buffer (137 mM NaCl, 2.7 mM KCl, 1 mM MgCl₂, 0.5 mM Na₃VO₄, 1% Triton X-100, 10% glycerol, 20 mM Tris pH 8.0, 10 g/ml leupeptin, 0.2 mM PMSF, 10 mM NaF, 10 μg/ml aprotinin). Lysates were rotated for 30 min at 4° C. and centrifuged at 12,000×g for 10 min at 4° C. The supernatant was removed and protein concentration was determined. Muscle lysates (150 μg of protein) were subjected to SDS/PAGE (7.5% gels), transferred to PVDF-membranes, and immunoblotted with a specific antibody against DGKδ. Proteins were visualized by ECL and quantified by densitometric scanning.

In diabetic GK rats, the protein levels of DGKδ in skeletal muscle are reduced. Treating the animals with phlorizin, an agent normalizing glucose homeostasis in the blood, results in an increase of DGK content in the muscle to approximately the same values as measured in healthy Wistar rats, treated with phlorizin or untreated. These data support the findings from human skeletal muscle, where DGK expression was decreased in diabetic muscle, and supports the conclusion that DGKδ is part of the physiological consequences of diabetes.

(b) Total DGK Activity

Portions of the gastrocnemius skeletal muscle (approximately 30 mg) were crushed in liquid nitrogen, homogenized by sonication in ice-cold buffer containing 20 mM Tris-HCl, pH 7.5, 250 mM sucrose, 1 mM EDTA, 1 mM EGTA, 1 mM dithiothreitol (DTT), 1 mM PMSF, a 20 μg/ml concentration of each: leupeptin, pepstatin, aprotinin, and soybean trypsin inhibitor and centrifuged at 12,000×g for 10 min at 4° C. The supernatant was collected and protein concentration was determined.

An octyl glucoside/PS mixed-micelle assay of total DGK activity was performed using [γ-³²P] ATP. The assay mixture contained 50 mM MOPS, pH 7.2, 100 mM NaCl, 20 mM MgCl₂, 1 mM EGTA, 1 mM DTT, 2 mM diacylglycerol, 3.5 mM phosphatidylserine, 75 mM octyl-glucopyranoside, 500 μM [γ-³²P] ATP, and 20 μg of muscle protein in a volume of 100 μl. The reaction was initiated by the addition of [γ-³²P] ATP (20-30 μCi/μmol) and processed for 15 min at 24° C. The reaction was terminated with 0.5 ml of MeOH, 0.5 ml of CH₃Cl, 0.3 ml of 1% perchloric acid, and 25 μg of phosphatidic acid as carrier. The lower phase of each sample was washed twice with 1 ml of 1% perchloric acid and loaded on thin layer chromatography plate (20×20 cm Silica 60A plates; Whatman). The plates were developed in (325:75:25) CH₃Cl/MeOH/HOAc. The region containing phosphatidic acid was quantified by phosphoimaging and densitometry.

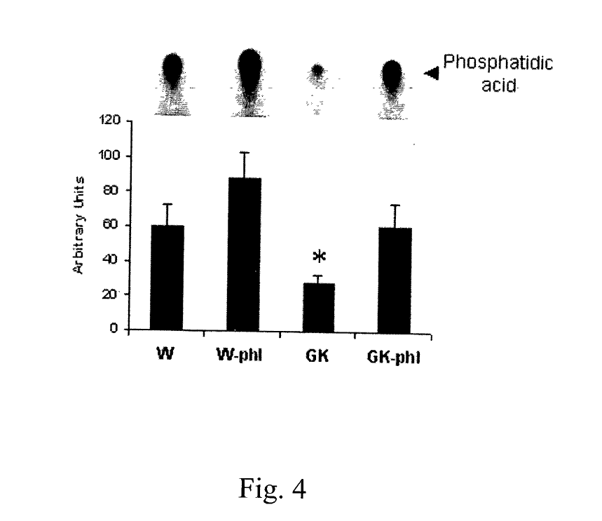

The results (FIG. 4) show an increase of total DGK activity in GK rat muscle upon phlorizin treatment to a level measured in muscle from healthy Wistar rats. This indicates that not only DGKδ expression, but also total DGK activity is affected under diabetic conditions, and can be normalized by phlorizin treatment.

### Example 4

### Inhibition of DGK Reduces Insulin-Stimulated Glucose Transport

The influence of total DGK activity on insulin-stimulated glucose uptake was assessed by measurements on epitrochlearis muscle from Wistar rats using a specific inhibitor for diacylglycerol kinase, DGK inhibitor II™ (Jang, Y. et al. (2000) Biochem. Pharmacol. 59: 763-772).

Media were prepared from a pre-gassed (95% O₂/5% CO₂) Krebs Henseleit buffer (KHB) containing 5 mM glucose and 0.1% BSA (RIA grade). Epitrochlearis muscles were incubated (20 min) in a shaking water bath (30° C.) in 2 ml of KHB, supplemented with 20 mM mannitol, with or without 2.4 nM insulin and 25 μM DGK-II inhibitor (Calbiochem). Muscles were transferred to KHB containing 8 mM 3-O-methyl[³H]glucose (438 μCi/mmol; 1 Ci=37 kBq) and 12 mM [¹⁴C]mannitol (42 μCi/mmol) and incubated (10 min) with or without insulin and 25 μM of DGKII inhibitor. 3-O-methyl-glucose transport activity was assessed as described (Wallberg-Henriksson et al. (1987) J. Biol. Chem. 262: 7665-7671) and expressed as micromoles per milliliter of intracellular water per hour.

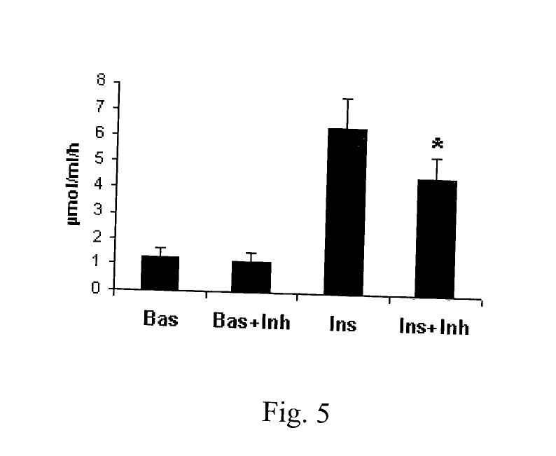

The results (FIG. 5) indicate that basal glucose transport in Wistar rats was not affected by treatment with DGK inhibitor II™. However, with the inhibitor, insulin-stimulated glucose transport was decreased by approximately 30%. The DGK inhibitor II™ reduced total DGK activity by 70% (data not shown). Reduction of insulin-stimulated glucose transport by DGK inhibitor II™ leads to the conclusion that DGK activity is involved in insulin signaling to glucose uptake. Since DGKδ is the predominant isoform in skeletal muscle (Sakane et al., J Biol Chem 271: 8394-8401, 1996) these effects can be attributed to DGKδ.

### Example 5

### Screening for DGKδ-Interacting Proteins

In order to assay for DGKδ-interacting proteins, the interaction trap/two-hybrid library screening method can be used. This assay was first described in Fields et al. (1989) Nature 340, 245. A protocol is published in Current Protocols in Molecular Biology 1999, John Wiley & Sons, NY and Ausubel, F. M. et al. (1992) Short protocols in molecular biology, fourth edition, Greene and Wiley-interscience, NY. Kits are available from Clontech, Palo Alto, Calif. (Matchmaker Two-Hybrid System 3).

A fusion of the nucleotide sequences encoding all or partial DGKδ and the yeast transcription factor GAL4 DNA-binding domain (DNA-BD) is constructed in an appropriate plasmid (i.e. pGBKT7) using standard subcloning techniques. Similarly, a GAL4 active domain (AD) fusion library is constructed in a second plasmid (i.e. pGADT7) from cDNA of potential DGKδ-binding proteins (for protocols on forming cDNA libraries, see Sambrook et al. 1989, Molecular cloning: a laboratory manual, second edition, Cold Spring Harbor Press, Cold Spring Harbor, N.Y.). The DNA-BD/DGKδ fusion construct is verified by sequencing, and tested for autonomous reporter gene activation and cell toxicity, both of which would prevent a successful two-hybrid analysis. Similar controls are performed with the AD/library fusion construct to ensure expression in host cells and lack of transcriptional activity. Yeast cells are transformed (ca. 10⁵ transformants/mg DNA) with both the DGKδ and library fusion plasmids according to standard procedure (Ausubel, et al., 1992, Short protocols in molecular biology, fourth edition, Greene and Wiley-interscience, NY). In vivo binding of DNABD/DGKδ with AD/library proteins results in transcription of specific yeast plasmid reporter genes (i.e., lacZ, HIS3, ADE2, LEU2). Yeast cells are plated on nutrient-deficient media to screen for expression of reporter genes. Colonies are dually assayed for β-galactosidase activity upon growth in Xgal (5-bromo-4-chloro-3-indolyl-β-D-galactoside) supplemented media (filter assay for β-galactosidase activity is described in Breeden et al., (1985) Cold Spring Harb. Symp. Quant. Biol. 50: 643). Positive AD-library plasmids are rescued from transformants and reintroduced into the original yeast strain as well as other strains containing unrelated DNA-BD fusion proteins to confirm specific DGKδ/library protein interactions. Insert DNA is sequenced to verify the presence of an open reading frame fused to GAL4 AD and to determine the identity of the DGKδ-binding protein.

### OTHER EMBODIMENTS

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method for identifying an agent that modulates the ability of a DGKδ polypeptide to modulate insulin-stimulated glucose uptake in a cell, the method comprising:
contacting a DGKδ polypeptide with a candidate agent; and
determining the effect of the candidate agent on the ability of the DGKδ polypeptide to modulate insulin-stimulated glucose uptake in a cell.

2. The method of claim 1, wherein the DGKδ polypeptide is a mammalian DGKδ polypeptide.

3. The method of claim 1, wherein the method comprises determining the effect of the candidate agent on the ability of the DGKδ polypeptide to increase insulin-stimulated glucose uptake in the cell.

4. A method for identifying an agent that modulates insulin-stimulated glucose uptake in a cell, the method comprising:
contacting a DGKδ polypeptide with a candidate agent;
determining whether the candidate agent binds to the DGKδ polypeptide; and
determining the effect of the candidate agent on insulin-stimulated glucose uptake in a cell.

5. The method of claim 4, wherein the DGKδ polypeptide is a mammalian DGKδ polypeptide.

6. The method of claim 4, wherein the candidate agent increases insulin-stimulated glucose uptake in the cell.

7. A method for identifying an agent that modulates insulin-stimulated glucose uptake in a cell, the method comprising:
contacting a nucleic acid molecule encoding a DGKδ polypeptide with a candidate agent;
determining whether the candidate agent activates expression of the nucleic acid molecule; and
determining the effect of the candidate agent on insulin-stimulated glucose uptake in a cell.

8. The method of claim 7, wherein the DGKδ polypeptide is a mammalian DGKδ polypeptide.

9. The method of claim 7, wherein the candidate agent increases insulin-stimulated glucose uptake in the cell.

10. A method for identifying a modulator of insulin-stimulated glucose uptake, the method comprising:
providing a cell expressing a recombinant DGKδ polypeptide;
exposing the cell to a candidate agent; and
measuring insulin-stimulated glucose uptake in the cell in the presence of the candidate agent,
wherein altered insulin-stimulated glucose uptake in the cell in the presence of the candidate agent compared to the absence of the candidate agent indicates that the candidate agent is a modulator of insulin-stimulated glucose uptake.

11. A method for modulating insulin-stimulated glucose uptake in a cell, the method comprising contacting a cell with an amount of a compound effective to modulate expression or activity of a DGKδ polypeptide and thereby modulate insulin-stimulated glucose uptake in the cell.

12. The method of claim 11, wherein the compound increases expression of the DGKδ polypeptide.

13. The method of claim 11, wherein the compound increases kinase activity of the DGKδ polypeptide.

14. A method for treating or preventing diabetes, the method comprising:
selecting an individual that has or is at risk of having diabetes; and
administering to the individual a compound that increases expression or activity of a DGKδ polypeptide in an amount effective to treat or prevent diabetes.

15. The method of claim 14, wherein the individual is diagnosed as having diabetes.

16. A method of diagnosing the presence or risk of diabetes in a mammalian subject, the method comprising:
detecting the level of DGKδ expression in a cell or tissue derived a mammalian subject; and
comparing the level of DGKδ expression to a healthy subject,
wherein a decreased level of DGKδ expression, as compared to a healthy subject, indicates the presence or risk of diabetes in the mammalian subject.

17. The method of claim 16, wherein the method comprises detecting the level of a nucleic acid encoding a DGKδ polypeptide.

18. The method of claim 16, wherein the method comprises detecting the level of a DGKδ polypeptide.
